(19) United States
(12) Plant Patent Application Publication
(10) Pub. No.: US 2003/0088897 P1
(43) Pub. Date: May 8, 2003

(54) Peach tree named 'FA 42'

(76) Inventors: Annette Bjorge, Coloma, MI (US); Randy Bjorge, Coloma, MI (US)

Correspondence Address: Wanda Heuser Gale, International Plant Management, Inc., 55826 60th Avenue, Lawrence, MI, 49064, US

(21) Appl. No.: 10/004,256

(22) Filed: Nov. 2, 2001

(51) Int. Cl. A01H 5/00

(52) U.S. Cl. PLT/198

(57) ABSTRACT

A new peach variety, Prunus persica, (hereinafter referred to as ‘FA 42 peach’) which was developed in 1981 by Annette and Randy Bjorge in a breeding program at Fruit Acres Farms in Coloma, Mich., having the following combination of unique and desirable features:

1. A substantially large, round fruit with an attractive bright red blush covering 75 to 80% of the fruit at maturity.

2. A fruit maturing late in the season, 7 days after ‘Fayette’ or 45 days after ‘Redhaven’.

3. A fruit with excellent storage and shipping qualities.

4. A fruit with flesh that does not brown when cut.

### FIELD OF THE INVENTION

A new and distinct variety of peach tree originating as a cross of ‘Autumnglo’ ‘Fayette’ varieties of Prunus persica hereinafter referred to as ‘FA 42’ peach. This sport is unique from its parents because the fruit is both highly colored and round and the tree has good bud and wood hardiness. ‘FA 42’ ripens later than both parents, 6 days after ‘Autumnglo’ and 7 days after ‘Fayette’ with large, 75 to 80% blushed red fruit.

### SUMMARY OF THE INVENTION

This new and distinct variety of peach tree was developed in a breeding program in 1981 by Annette and Randy Bjorge of Coloma, Mich. It resulted as a cross of ‘Autumnglo’ and ‘Fayette’, both unpatented peach cultivars. The new variety was noticed because it ripened very late in the season with a high color factor and large size.

The new variety was budded on Bailey peach seedling in the summer of 1988 and planted for further testing on Fruit Acres Farms, Coloma, Mich. In the summer of 1992, ‘FA 42’ was budded on Halford peach seedling. In the spring of 1993, these trees were sent to the following cooperating testers under testing agreements: Rutgers Snyder Research Farm, Pittstown, NJ; Lee Spencer, Boyertown, Pa; Adams County Nursery, Aspers, Pa; Kenneth Kauffman, Bird-in-Hand, Pa; Thomas Benton, Chester, N.J.; Clemson University, Clemson, S.C.; Arnold Klug, Berrien Center, Mich.; North Carolina State University, Raleigh, N.C.; Rutgers Cooperative Extension, Clayton, N.J.; SW Michigan Research & Extension Center, Benton Harbor, Mich.; and The University Of Arkansas, Clarksville, Ak. The reports from the cooperating testers showed that the new variety remained true to name and showed considerable commercial promise as a peach for the late harvest season.

### COMPARISON TO PRIOR ART

‘FA 42’ ripens vary late in the commercial peach season with a large sized fruit and a very high degree of red skin color. This contrasts to ‘Fayette’, (an unpatented selection) which ripens 13 days earlier and is much less highly colored. ‘FA 42’ ripens 6 days after ‘Autumnglo’ (an unpatented selection) and is more highly colored with a lighter degree of pubescence. The new variety ripens 6 days after ‘Encore’ (U.S. Plant Pat. 4,572) and has a much higher degree of red skin color. ‘FA 42’ ripens 15 days after PF 27A (U.S. Plant Pat. 9,939). ‘FA 42’ ripens 2 days after “Laurol’ (U.S. Plant Pat. 8,558) and has a bright red skin color contrasting with the orangered red of ‘Laurol’. ‘FA 42’ also has a much greater degree of bud and wood hardiness than ‘Laurol’.

### BRIEF DESCRIPTION OF THE DRAWINGS

The accompanying photographs show typical specimens of the new variety as depicted in color as nearly true as is reasonably possible in color illustrations of this character. These specimens were obtained at Coloma, Berrien County, Mich.

FIG. 1 illustrates the coloration of the fruit of the new variety in comparison with the coloration of ‘Fayette’ and ‘Encore’ (U.S. Plant Pat. 4,572). ‘Encore’, picked on September 17, is pictured on the top; ‘FA 42’ picked on September 24, is pictured in the middle segment; ‘Fayette’, picked on September 11, is shown in the bottom section. All three varieties were grown on the same farm in Coloma, Berrien County, Mich..

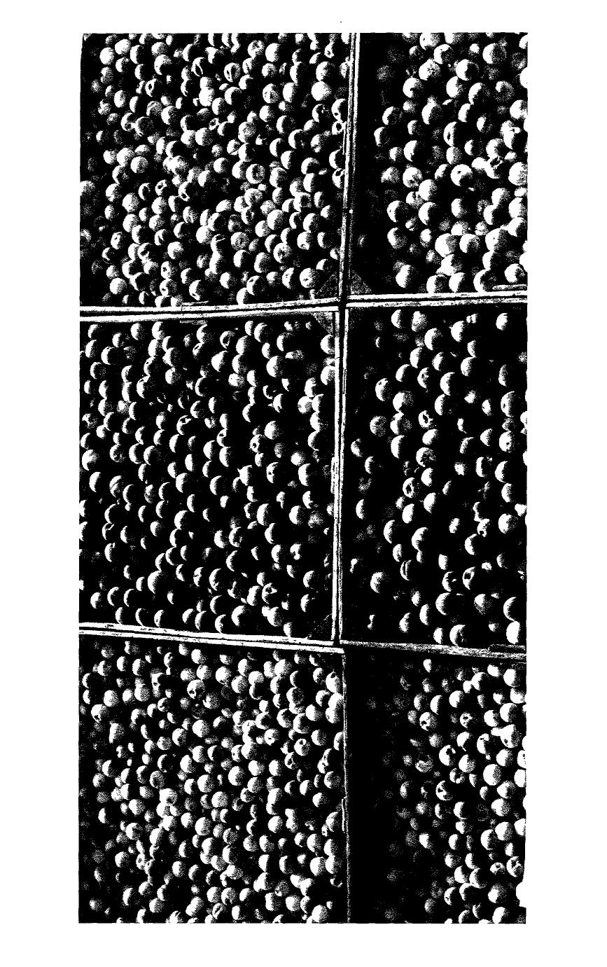

FIG. 2 illustrates several fruit of the new variety at maturity.

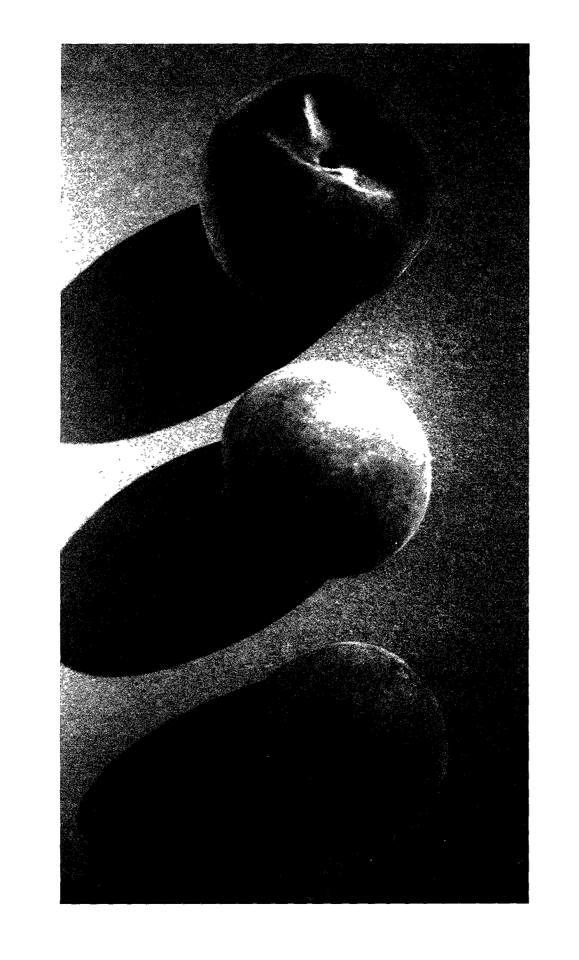

FIG. 3 illustrates sections, leaves, and pits of the new variety at maturity.

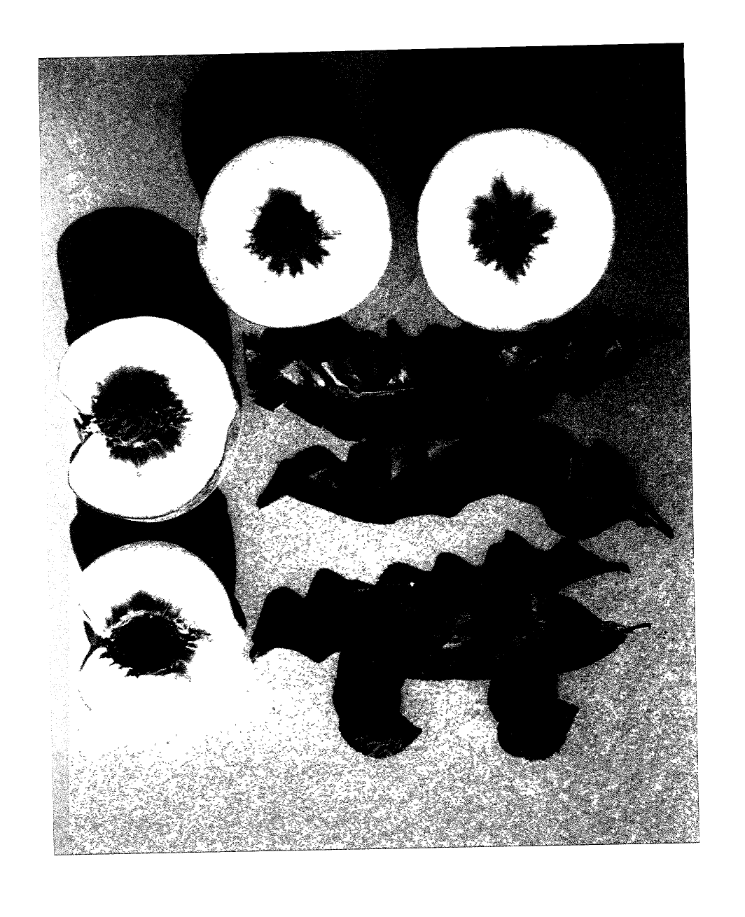

### BOTANICAL DESCRIPTION OF THE PLANT

A detailed description of the ‘FA 42’ cultivar follows using the Royal Horticultural Society of London Colour Chart for color identification except where general color terms are sufficient.

Parentage: ‘Autumnglo’×‘Fayette’.

Locality of the original discovery and observations: Coloma, Berrien County, Michigan.

Tree:

Age of specimen.—5 years.

Height.—3M.

Width.—4M.

Vigor.—quite vigorous under normal growing conditions.

Average growth per year.—45 to 65 cm on terminal branches.

Density.—medium open.

Form.—vase shaped, average of 3 major scaffold branches with 12 to 14 laterals.

Production.—average for a freestone peach, usually 3 to 4 bushels per tree per year.

Bearing.—Annual.

Disease resistance.—moderate susceptibility to Bacterial Spot, (Xanthomonas campestris) and peach canker (Cytospora leucostoma and Cytospora cincta). Both have been seen in test plantings. No specific testing for disease susceptibility has been preformed.

Cold hardiness.—quite hardy, hardier than Redhaven, winter bud hardiness threshold averages −14° to −16° F., winter wood hardiness threshold averages −18° to −20° F.

Trunk:

Size.—12.5 cm at 15 cm above ground level.

Surface.—smooth with rough vertical striations and horizontal lenticels.

Lenticels.—length 12 to 15 mm, width 1 mm, color Grayed Orange 167A.

Striations.—length 8 to 10 mm, width 0.5 mm, color Grayed White 156D. 1 Year Branches: surface smooth, length 45 to 65 cm, diameter 1 mm, color Grayed Orange 167A, average angle to 2-year branch 50°. 2 Year Branches: surface smooth, length 45 to 60 mm, diameter 7 mm, color Grayed Orange 175C, average angle to 3-year branch 45°. 3 Year Branches: smooth surface, length 220cm, diameter 10 mm, color Grayed Orange 177C, average angle to 4 year scaffold 45°. 3 Year lenticels: length 1.5 mm, width 0.5 mm, color Grayed Orange 167D. 4 Year Scaffolds: slightly rough surface, diameter 12 mm, color Grayed Orange 167 B. 4 year Lenticels: length 6 mm, width 0.75 mm, color Grayed Orange 167 B.

Leaf Buds:

Size.—dormant length 2 mm, width 1 mm.

Bud scale color (dormant).—Grayed Orange 178B, bud tip color Grayed White 156A.

Placement.—tightly applied to branch.

Internode Distance.—22 mm.

Leaves:

Size.—length 85 mm, width 18 mm at widest point.

Form.—lanceolate, tip sharply pointed.

Thickness.—medium thin.

Texture.—crisp.

Margin.—roundly crenate.

Adaxile leaf surface.—Yellow Green 146A.

Abaxile leaf surface.—Yellow Green 146B.

Veination.—alternate, adaxial midvein color Yellow Green 153A, adaxial midvein color Yellow Green 158B.

Petiole.—length 5 to 6 mm, width 1.3 mm.

Petiole Groove.—depth 0.1 mm, extending entire length of petiole.

Glands.—reniform, 5 to 9 on the base of most leaves. Generally located on the upper petiole, occasionally on the lower leaf.

Gland Color.—Red Purple 59B.

Flowers:

Blooming period.—early.

Presentation.—non showy.

Fruit:

Maturity when described.—shipping ripe.

Date of first picking.—September 24, 45 days after ‘Redhaven’, 7 days after ‘Fayefte’, 2 days after ‘Laurol’.

Date of last picking.—September 31, may vary by 2 to 3 days due to temperature.

Size.—large, diameter 32 cm, length 29 cm.

Form.—round, even shoulders, slight bulge on pistil point, tip flat to slightly indented.

Stem cavity.—5 mm deep, 9 mm wide, stem abscises cleanly.

Skin:

Thickness.—medium.

Texture.—slightly tough

Tendency to crack.—very slight.

Color.—dark red blush of Red 53A on 75 to 80% of the surface, middle blush of Red 47B, background color of yellow 11A.

Flavor.—good, non-bitter.

Pubescence.—very low, very fine.

Flesh:

Texture.—firm, dense, melting.

Acidity.—medium high.

Flavor.—excellent, very good acid/sugar balance.

Aroma.—very high.

Fibers: few, very fine.

Color.—clear, Yellow 11C, non-browning.

Coloration at the pit.—Red 46A at interface with pit bleeding to Red 47B in the flesh. Depth of coloring into the flesh 1.5 to 2 mm.

Eating quality.—Excellent.

Stone:

Size.—length 16 mm, width 11 mm.

Form.—elongated, elliptical with a prominent tip.

Apex.—sharply pointed, point extends 2.5 mm from body of stone.

Sides.—even.

Base.—flat, slightly tilted at abscission layer with stem.

Surface.—deeply furrowed.

Dorsal groove margin.—clean, slightly raised.

Color.—Grayed Orange 165A.

Type.—freestone.

Tendency to crack.—slight, may crack under wet, hot, conditions. Use: Fresh market. Shipping quality: Excellent, can be graded on a commercial peach grader and shipped via truck. Keeping quality: Excellent, will store for up to 2 months in common storage with minimal loss of flavor and color.

I claim:

1. A new and distinct variety of peach tree, Prunus persica, substantially as herein shown and described, characterized particularly as to novelty by the unique combination of vigorous, regular bearing tree producing freestone fruits having with non-browning flesh and large, round, firm fruits with 75 to 80% blushed red skin color at maturity which occurs 45 days after the maturity of ‘Redhaven’ peach.
